(19) United States
(12) Patent Application Publication
(10) Pub. No.: US 2013/0041398 A1
(43) Pub. Date: Feb. 14, 2013

(54) Dilator

(71) Applicants: James Goddard, Pepperell, MA (US); Isaac Ostrovsky, Wellesley, MA (US); David Salto, Hopedale, MA (US)

(72) Inventors: James Goddard, Pepperell, MA (US); Isaac Ostrovsky, Wellesley, MA (US); David Salto, Hopedale, MA (US)

(21) Appl. No.: 13/568,713

(22) Filed: Aug. 7, 2012

Related U.S. Application Data
(60) Provisional application No. 61/521,066, filed on Aug. 8, 2011.

(51) Int. Cl. A61M 29/00

(52) U.S. Cl. 606/191

(57) ABSTRACT

A device for dilating a body passage includes an inner element including a shaft portion extending longitudinally from a proximal end to a distal end and an enlarged portion situated in the distal end and an outer sleeve extending longitudinally from a proximal end to a distal end and including a lumen extending therethrough. The lumen is sized and shaped to slidably accommodate the shaft portion of the inner element such that the device is movable between an insertion configuration, in which the enlarged portion extends distally past the distal end of the outer sleeve, and an expanded configuration, in which the distal end of the outer sleeve receives the enlarged portion of the inner element therein.

### PRIORITY CLAIM

The present application claims priority to U.S. Provisional Application Ser. No. 61/521,066 entitled “Dilator” filed on Aug. 8, 2011, the entire disclosure of which is incorporated herewith by reference.

### BACKGROUND

Many gynecological procedures require dilation of the cervix. Known methods for dilating the cervix include inserting increasingly larger rods or tubes into the cervical canal until a desired dilation has been obtained. Such systems and methods, however, may damage the uterine cavity if the rods or tubes are inserted too far. In addition, the process may be time consuming since a physician or other user must individually insert and remove each of the rods or tubes.

### SUMMARY OF THE INVENTION

The present invention relates to a device for dilating a body passage, comprising an inner element including a shaft portion extending longitudinally from a proximal end to a distal end and an enlarged portion situated in the distal end of the shaft portion and an outer sleeve extending longitudinally from a proximal end to a distal end and including a lumen extending therethrough, the lumen sized and shaped to slidably accommodate the shaft portion of the inner element such that the device is movable between an insertion configuration, in which the enlarged portion extends distally past the distal end of the outer sleeve, and an expanded configuration, in which the distal end of the outer sleeve receives the enlarged portion of the inner element therein.

### BRIEF DESCRIPTION OF THE DRAWINGS

FIG. 1 shows a cross-sectional side view of a device according to a first exemplary embodiment of the present invention, in a first configuration;

FIG. 2 shows a cross-sectional side view of the device of FIG. 1, in a second configuration;

FIG. 3 shows a side view of a device according to another exemplary embodiment of the present invention, in a first configuration;

FIG. 4 shows a side view of the device of FIG. 3, in a second configuration;

FIG. 5 shows a perspective view of a device according to yet another embodiment of the present invention;

FIG. 6 shows a side view of a dilating member of the device of FIG. 5;

FIG. 7 shows a longitudinal cross-section of a distal portion of the device of FIG. 5;

FIG. 8 shows a cross-sectional view of an outer ring of the dilating member of the device of FIG. 7;

FIG. 9 shows a side view of a device according to another exemplary embodiment of the present invention;

FIG. 10 shows an enlarged side view of a distal portion of the device of FIG. 9, in a first configuration;

FIG. 11 shows an enlarged view of the distal portion of the device of FIG. 9, in a second configuration; and

FIG. 12 shows a cross-sectional view of the device of FIG. 9, along line A-A.

### DETAILED DESCRIPTION

The present invention may be further understood with reference to the following description and the appended drawings, wherein like elements are referred to with the same reference numerals. The present invention relates to the treatment of body passages and, in particular, relates to the dilation of a body passage. Exemplary embodiments of the present invention describe a device for dilating a cervix or other body passage. It should be noted that the terms “proximal” and “distal,” as used herein, are intended to refer to a direction toward (proximal) and away from (distal) a user of the device.

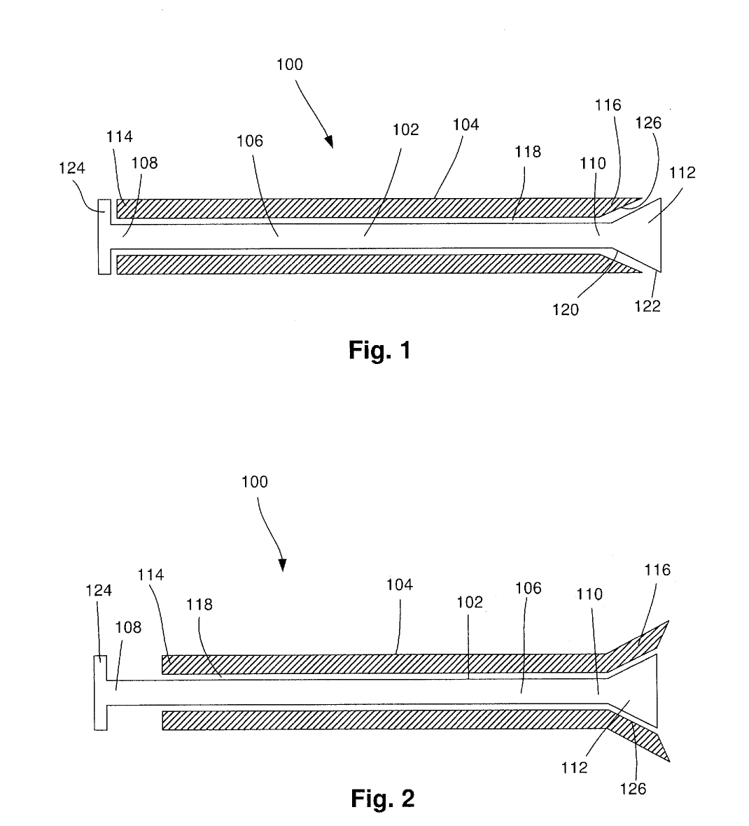

As shown in FIGS. 1-2, a device 100 according to a first exemplary embodiment comprises an inner element 102 extending through a lumen 103 in an outer sleeve 104 sized and shaped to be inserted into a body passage such as, for example, the cervix. The inner element 102 is longitudinally movable within the lumen 1218 to move the device 100 between an insertion/withdrawal configuration shown in FIG. 1 and a dilation configuration for dilating the body passage. The inner element 102 includes an enlarged portion 112 which, in the insertion configuration, is received in an enlarged distal end of the lumen 118 so that, proximal movement of the inner element 102 relative to the outer sleeve 104 draws the enlarged portion 112 into the proximal, reduced diameter portion of the lumen 118, expanding the distal end 116 of the outer sleeve 104 to the expanded configuration.

The inner element 102 includes a shaft 106 extending longitudinally from a proximal end 108 to a distal end 110 and an enlarged portion 112 at the distal end 110 of the shaft 106. The enlarged portion 112 flares distally outward from the distal end 110. In a preferred exemplary embodiment, the enlarged portion 112 is substantially conical, increasing in diameter from a proximal end 120 to a distal end 122. It will be understood by those of skill in the art, however, that the enlarged portion 112 may be any of a variety of shapes so long as the enlarged portion 112 has a cross-sectional area larger than that of the shaft 106 and the reduced diameter proximal portion of the lumen 118 and increases from the proximal 120 to the distal end 122. The inner element 102 may also include a handle 124 at the proximal end 108 of the shaft 106 for moving the inner element 102 relative to the outer sleeve 104.

The outer sleeve 104 also extends longitudinally from a proximal end 114 to a distal end 116 with the lumen 118 extending therethrough. The lumen 118 is sized and shaped to accommodate the shaft 106 of the inner element 102 so that the outer sleeve 104 surrounds the shaft 106 of the inner element 102. In the insertion configuration, the enlarged portion 112 extends distally past the distal end 116 of the outer sleeve 116 and the handle 124 is proximal of the proximal end 114. The lumen 118 may include an angled surface 126 at the distal end 116 configured to interface with the enlarged portion 112 such that, in the expanded configuration, the enlarged portion 112 is slidably received within the distal end 116 of the outer sleeve 104. As the enlarged portion 112, is drawn proximally into the lumen 118, the distal end 116 expands to accommodate the enlarged portion 112 therein. In one exemplary embodiment, the distal end 116 of the outer sleeve 104 may expand to approximately between 11 mm and 18 mm. At least the distal end 116 of the outer sleeve 104 is preferably formed of an elastic material that expands as it receives the enlarged portion 112 of the inner element 102 therein. For example, at least the distal end 116 may be formed of materials such as silicone, polyurethane or a PE/EVA blend. It will be understood by those of skill in the art, however, that other portions of the outer sleeve 104 may also be formed of one of these materials.

In use, the device 100 is inserted into the body passage (e.g., cervix) in the insertion configuration, until the enlarged portion 112 at the distal end 110 of the shaft 106 is within a body cavity (e.g., uterus). Once the device 100 has been properly positioned within the body passage, the inner element 102 is drawn proximally relative to the outer sleeve 104 via, for example, the handle 124, so that the enlarged portion 112 is drawn into the lumen 118 and the distal end 116 of the outer sleeve 104 expands to accommodate the enlarged portion 112 therein. The expansion of the outer sleeve 104 dilates the body passage, as desired.

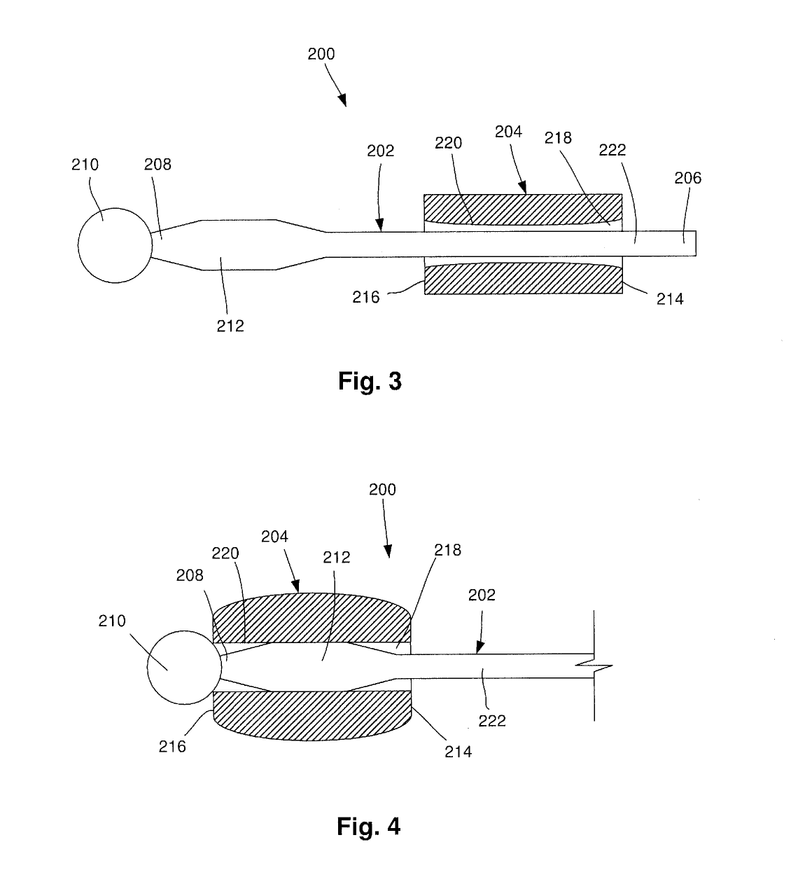

As shown in FIGS. 3-4, a device 200 according to a third embodiment of the invention comprises a shaft 202 and a dilating member 204 slidable over the shaft 202 to move the device from an insertion configuration, as shown in FIG. 3, to an expanded configuration, as shown in FIG. 4, to dilate the body passage. The shaft 202 extends longitudinally from a proximal end 206 to a distal end 208 and includes a stop 210 at the distal end 208. Immediately proximal the stop 210, the shaft 202 includes an increased diameter portion 212 which, when the dilating member 204 is slid thereover, moves the dilating member 204 from the insertion configuration to the expanded configuration. The stop 210 may be formed as a ball having a diameter larger than that of the shaft 202. It will be understood by those of skill in the art, however, that the stop 210 may be any of a variety of shapes so long as the stop 210 is sized to prevent the dilating member 204 from moving distally therepast. Furthermore, those skilled in the art will recognize that the outer diameter of the stop 210 and the increased diameter portion 212 are less than a desired diameter of dilation of the body passage so that the shaft 202 may be more easily inserted through the body passage.

In an exemplary embodiment, the device 200 may be sized and shaped to dilate the cervix. The outer diameter of the increased diameter portion 212, in this embodiment, may be approximately 2.25 mm to facilitate insertion of the distal end 208 of the shaft 202 through the cervix with this diameter tapering to the outer diameter of the shaft 202 at the proximal and distal ends of the increased diameter portion 212. The outer diameter of the remaining portion 222 of the shaft 202 may range from between 1.0 mm to 1.25 mm. The outer diameter of the increased diameter portion 212 may be substantially constant along a portion thereof between the tapered proximal and distal ends thereof. In another embodiment, the outer diameter of the increased diameter portion 212 may remain substantially constant along its length without the tapered ends. Alternatively, the outer diameter of the increased diameter portion 212 may increase gradually along a curve from the proximal end toward a midpoint of maximum diameter and then may extend along the curve back toward the outer diameter of the remaining portion 222 of the shaft 202 at its distal end.

The dilating member 204 may be, for example, a substantially tubular radially expansible member extending from a proximal end 214 to a distal end 216. The dilating member 204 includes a channel 218 extending therethrough with an inner diameter that varies along at least a first portion of a length thereof to amplify the expansion of the dilating member 204 as the increased diameter portion 212 is inserted therein. A length of the first portion of the dilating member 204 is selected to be substantially equal to a length of the body passage to be dilated. The channel 218 is sized and shaped to slidably receive the shaft 202, having a minimum inner diameter slightly larger than an outer diameter of the remaining portion 222 of the shaft 202. At least the first portion of the dilating member 204 is formed of an elastic material so that, as the dilating member 204 is moved over the increased diameter portion 212, the dilating member 204 expands to accommodate the increased diameter portion 212 therein, thereby moving from the insertion configuration to the expanded configuration. As the diameter of the channel 218 is larger at the distal end 216 of the channel 218, the increased diameter portion 218 may be slid therealong and into the channel 218. For example, an inner surface 220 of the channel 218 may be curved or angled radially inward. Similarly, the proximal end 214 of the channel 218 may be curved or angled radially inward to facilitate insertion of the increased diameter portion 212 thereinto from the proximal side of the first portion of the dilating member 204. In a device according to an alternate embodiment of the invention, an inner diameter of the channel 218 may be constant along an entire length of the first portion of the dilating member 204. However, in this embodiment, as would be understood by those skilled in the art the amplification of the dilation of the dilating member 204 as the increased diameter portion 212 enters the restricted diameter portion of the channel 218 will not be achieved.

Where the dilating member 204 is used to dilate the cervix, the dilating member 204 preferably has an outer diameter of approximately 3.5 mm to 4.0 mm in the insertion/withdrawal configuration, to facilitate insertion into the cervix. In the expanded configuration, where the increased diameter portion 212 is received within the channel 218, the dilating member 204 is preferably expanded to a diameter of approximately 4.5 mm to 5.0 mm. It will be understood by those of skill in the art, however, that the dimensions of the dilating member 204, the restricted diameter of the channel 218 and the dimensions of the increased diameter portion 212 are exemplary only and that any of these dimensions may be altered as necessary to achieve the desired characteristics of a device in the insertion/withdrawal and/or expanded configurations. For example, additional dilating members 204, each with progressively larger outer diameters, may be used to steadily increase a dilation of the body passage.

In use, the shaft 202 is inserted through the body passage until the stop 210 moves distally past the distal opening of the body passage (e.g., distally beyond the distal cervical os into the uterus) into the body cavity (e.g., uterus). The dilating member 204 is then slid distally over the shaft 202 until the dilating member is positioned proximally of the increased diameter portion 212 of the shaft 202, within a portion of the body passage to be dilated. The user then pulls the shaft 202 proximally via, for example, the proximal end 206 to draw the increased diameter portion 212 into the channel 218 of the dilating member 204, which must be restricted at the same location. The user may draw the shaft 202 proximally until the stop 210 contacts the end 216 of the dilator 204. The stop 210 is preferably positioned so that, at this point, the restricted diameter portion of the channel 218 is received over the increased diameter portion 212 of the dilating member 204 to expand the dilating member 204 and dilate the body passage as desired. Once the stop 210 has reached the distal end 216 of the dilating member 204, both the dilating member 204 and the shaft 202 are withdrawn. If desired, after this first dilation of the body passage, the shaft 202 may be reinserted into the body passage and a second dilating member with a larger outer diameter may be slid over the shaft 202 and expanded, as described above. This process may be repeated with increasingly larger dilating members until a desired dilation of the body passage has been achieved. The shaft 202 may also remain within the body passage so that each additional dilating member may be slid in and out thereover. It will be understood by those of skill in the art that the shaft 202 may act as a guide for each of the additional dilating members.

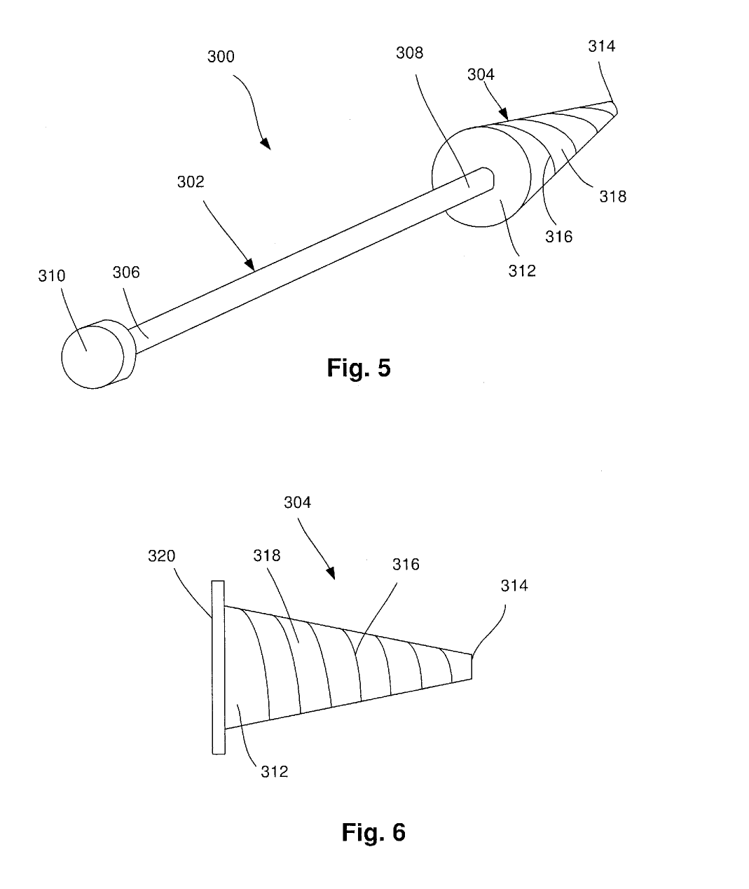

As shown in FIGS. 5-8, a device 300 according to a third exemplary embodiment of the present invention comprises a shaft 302 and a dilating member 304 that may be threaded into a body passage to dilate the body passage. As shown in FIG. 5, the shaft 302 extends longitudinally from a proximal end 306 to a distal end 308, the dilating member 304 attached to the distal end 308 of the shaft 302. The device 300 further comprises a handle 310 at the proximal end 306 for applying rotational force to the dilating member 304 at the distal end 308 of the shaft 302. Thus, the shaft 302 is torsionally stiff so that rotation of the handle 310 correspondingly rotates the dilating member 304 at the distal end 308 thereof.

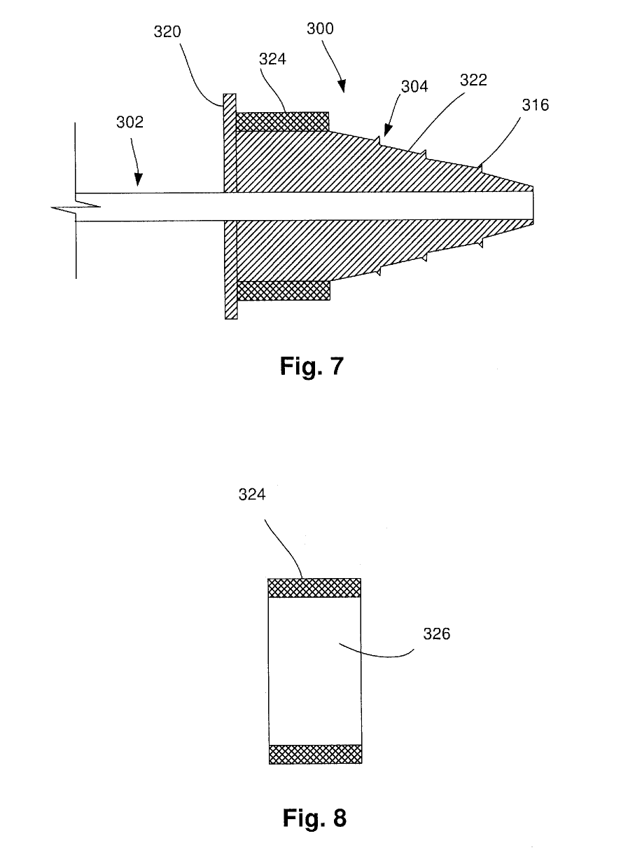

The dilating member 304, as shown in FIGS. 6-7 includes a body 322 tapering from a proximal end 312 to a distal end 314 and including a helical thread 316 about an outer surface 318 thereof. As shown in FIG. 6, the body 322 of the dilating element 304 is tapered (e.g., substantially conical) decreasing in diameter gradually from the proximal end 312 to the distal end 314. Where the dilating member 304 is to be used to dilate a cervix, for example, the proximal end 312 has a diameter of approximately 10 mm while the distal end 314 may have a diameter of approximately 2 mm. It will be understood by those of skill in the art, however, that the dilating member 304 may be formed of any tapering structural element and with any dimensions suitable to the body passage to be dilated. For example, the diameters of the proximal and distal ends 312, 314 are exemplary only and may be of any size, determined by a size of the body passage and a desired dilation thereof. The dimensions of the proximal end 312 may correspond to the desired dilation of the body passage while the distal end corresponds to an initial, undilated, size of the body passage. The dilating member 304 may also include a stop 320 at the proximal end 312 of the body 322 to prevent the dilating member 304 from being inserted too far into the body passage. The stop 320 extends radially outward from the proximal end 312 such that the stop 320 has a diameter larger than the proximal end 312 of the body 322 and a proximal opening of the body passage.

In use, the dilating member 304 is inserted into the body passage and advanced thereinto by rotating the dilating member 304 via the handle 310 at the proximal end 306 of the shaft 302. The thread 316 along with the tapered structure of the body 322 facilitate distal advancement of the dilating member 304 into the body passage via rotation of the device 300, thereby gradually dilating the body passage. The dilating member 304 is “threaded” into the body passage until the stop 320 abuts the proximal opening of the body passage and prevents further distal movement thereof.

As shown in FIGS. 7-8, the dilating member 304 may further comprise an outer ring 324 extending about the proximal end 312 of the body 322. The outer ring 324 is releasably coupled to the body 322 such that, once the dilating member 304 has been inserted into the body passage as desired, the body 322 may be released from the outer ring 324 and removed from the body passage. The outer ring 324 may, for example, be coupled to the body 322 via a keyway and slot mechanism to permit the outer ring 324 to be released upon insertion in the body passage. The body 322 may be removed by drawing the shaft 302, which is attached to the body 322, proximally out of the body passage. Thus, only the outer ring 324 remains in the body passage providing a working channel 326 extending therethrough. In one exemplary embodiment, the working channel 326 has a diameter of approximately 10 mm. It will be understood by those of skill in the art, however, that the working channel 326 may be any of a variety of desired sizes and shapes so long as the working channel 326 corresponds in size and shape to the proximal end 312 of the body 322.

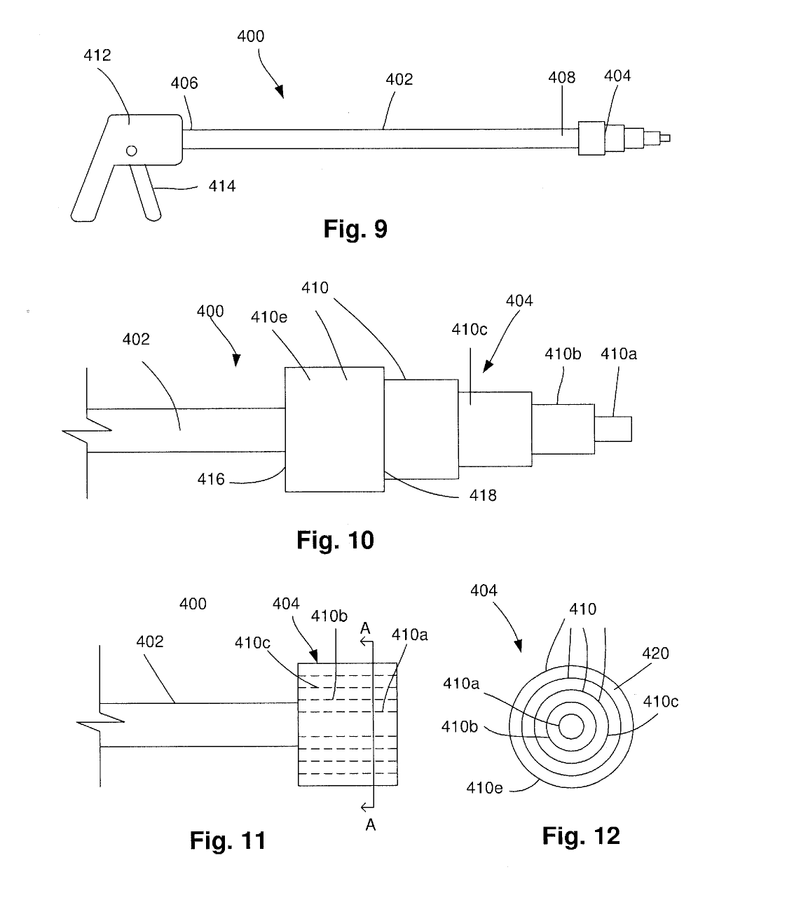

As shown in FIGS. 9-12, a device 400 according to a fourth exemplary embodiment of the present invention comprises a shaft 402 and a dilating member 404 formed of a plurality of dilating elements 410 slidable over one another to dilate a body passage via a single actuating motion. The shaft 402 extends longitudinally from a proximal end 406 to a distal end 408 and may include a handle 412 at the proximal end 406. The device 400 further comprises an actuating mechanism 414 which may be configured as, for example, a lever on the handle 412. Actuation of the actuating mechanism 414 via, for example, rotation of the lever, moves the dilating elements 410 from a first configuration for inserting the device 400 into the body passage to a second configuration for dilating the body passage.

Each of the dilating elements 410 may be substantially tubular and housed concentrically within one another, as shown in FIG. 12. The dilating elements 410 are coupled to one another such that the dilating elements 410 are slidable relative to one another and movable from the first configuration, as shown in FIG. 10, to the second configuration, as shown in FIG. 11. Each of the dilating elements 410 extends from a proximal end 416 to a distal end 418 and includes a lumen 420 extending. Each dilating element 410 varies in size so that each dilating element 410 is receivable within the lumen 420 of an adjacent dilating element 410, in size order. In the first configuration, the dilating member 404 is in an extended configuration so that only the proximal ends 416 of each of the dilating elements 410 are received within a distal end 418 of the immediately proximal concentric element 410. A distal-most concentric element 410a has an outer diameter smaller than an immediately proximal concentric element 410b and smaller than an outer diameter of the shaft 402. Similarly, the concentric element 410b has an outer diameter smaller than an immediately proximal concentric element 410. The device 400 may have any number of dilating elements 410 so that each concentric element 410 has an outer diameter smaller than an immediately proximal concentric element 410. It will be understood by those of skill in the art that the proximal-most concentric element 410e has the largest outer diameter, corresponding to a desired dilation of the body passage. Thus, the outer diameters of the dilating elements decrease from the proximal-most element 410e to the distal-most element 410a. In one exemplary embodiment, the distal-most dilating element 410a has an outer diameter of approximately 2 mm, each of the proximal dilating elements 410 gradually increasing in diameter so that the proximal-most dilating element 410e is approximately 10 mm. It will be understood by those of skill in the art that the dilating elements 410 may have any outer diameter so long as the dilating elements 410 gradually increase in size, from the distal-most element 410a to the proximal-most element 410e.

Upon actuation of the device 400 via the actuating mechanism 414, the dilating elements 410 are sequentially moved, into the second configuration, over one another to gradually dilate the body passage. First, the dilating element 410b immediately proximal the distal-most dilating element 410a is slid over the distal-most element 410a. Then, the dilating element 410c immediately proximal the dilating element 410b is slid over the dilating element 410b. Each immediately proximal dilating element 410 is sequentially moved over the immediately distal dilating element 410 until, the dilating member 404 is in the second compressed configuration, as shown in FIG. 10. In the compressed configuration, all the dilating elements 410 are substantially housed within the proximal-most element 410e.

In use, the device 400 is moved to the insertion configuration and the distal-most element 410a inserted into the body passage. Once the distal-most dilating element 410a has been positioned in the body passage, the actuating mechanism 414 is actuated so that each of dilating elements 410 proximal of the distal-most element 410a is sequentially moved over the distal-most element 410a, in size order. The immediately proximal element 410b is moved over the distal-most element 410a and into the body passage. Then, the element 410c immediately proximal the immediately proximal element 410cb is moved thereover into the body passage. Each of the remaining elements 410 is moved sequentially thereover, gradually dilating the body passage, until the dilating member 404 is in the second configuration and all the dilating elements are within the body passage.

It will be understood by those of skill in the art that various modifications and variations can be made in the structure and methodology of the present invention, without departing from the spirit or scope of the invention. Thus, it is intended that the present invention cover the modifications and variations of this invention provided that they come within the scope of the appended claims and their equivalents.

What is claimed is:

1. A device for dilating a body passage, comprising:
an inner element including a shaft portion extending longitudinally from a proximal end to a distal end and an enlarged portion situated in the distal end; and
an outer sleeve extending longitudinally from a proximal end to a distal end and including a lumen extending therethrough, the lumen sized and shaped to slidably accommodate the shaft portion of the inner element such that the device is movable between an insertion configuration, in which the enlarged portion extends distally past the distal end of the outer sleeve, and an expanded configuration, in which the distal end of the outer sleeve receives the enlarged portion of the inner element therein.

2. The device of claim 1, wherein the lumen of the outer sleeve includes an angled surface at the distal end portion thereof corresponding to an angled surface of the enlarged portion of the inner element.

3. The device of claim 1, wherein the inner element includes a handle at the proximal end thereof.

4. The device of claim 1, wherein, in the expanded configuration, the distal end of the outer sleeve expands to between 11 mm and 18 mm.

5. The device of claim 1, wherein the enlarged portion is substantially conical.

6. A device for dilating a body passage, comprising:
a shaft extending longitudinally from a proximal end to a distal end and including an enlarged portion proximate the distal end; and
a first dilating member extending from a proximal end to a distal end and including a channel extending therethrough, the first dilating member being sized and shaped for insertion into the body passage in an insertion configuration, the dilating member slidable over the shaft via the channel such that sliding the dilating member over the enlarged portion expands the dilating member to an expanded configuration.

7. The device of claim 6, further comprising:
a stop at the distal end of the shaft, the stop sized and shaped to prevent the first dilating member from being moved distally therepast.

8. The device of claim 7, wherein the stop is substantially spherical.

9. The device of claim 6, further comprising:
a second dilating member extending from a proximal end to a distal end and including a channel extending therethrough, the dilating member slidable over the shaft via the channel, the second dilating member having an outer diameter larger than an outer diameter of the first dilating member such that sliding the second dilating member over the expanded portion provides a second dilation of the body passage larger than the first dilation.

10. A device for dilating a body passage, comprising:
a shaft extending longitudinally from a proximal end to a distal end; and
a dilating member attached to the distal end of the shaft, the dilating member tapering from a proximal end to a distal end and including a helical threading along an outer surface thereof to facilitate rotative insertion into a body passage, a proximal end profile of the shaft corresponding to a desired amount of dilation of the body passage.

11. The device of claim 10, further comprising:
a stop at the proximal end of the dilating member extending radially outward therefrom to prevent insertion of the stop into the body passage.

12. The device of claim 10, further comprising:
an outer ring releasably coupled to the proximal end of the dilating member to provide a working channel.

13. The device of claim 12, wherein the outer ring is coupled to the proximal end of the dilating member via a keyway and slot mechanism.

14. The device of claim 10, further comprising:
a handle at the proximal end of the shaft to facilitate rotation of dilating member at the distal end of the shaft.

15. The device of claim 10, wherein the dilating member is substantially conical.

16. A device for dilating a body passage, comprising:
a shaft extending longitudinally from a proximal end to a distal end; and
a dilating member including a plurality of concentric elements movable relative to one another between an insertion configuration in which adjacent concentric elements are moved longitudinally away from one another and a dilating configuration, an outer diameter of the concentric elements increasing from a distal-most concentric element to a proximal-most concentric element so that the concentric elements are sequentially movable over an immediately distal element into the dilating configuration.

17. The device of claim 16, wherein the concentric elements distal of the proximal-most concentric elements are housed substantially therewithin in the dilating configuration.

18. The device of claim 16, further comprising:
a dilating actuator configured to sequentially move each of the concentric elements to the dilating configuration via a single actuating motion.

19. The device of claim 18, wherein the dilating actuator is a lever attached to a handle at the proximal end of the shaft.

20. The device of claim 18, wherein the diameter of the distal-most concentric element is smaller than a diameter of the shaft.
